(19) United States
(12) Patent Application Publication
(10) Pub. No.: US 2026/0229060 A1
(43) Pub. Date: Aug. 6, 2026

(54) ESTIMATION APPARATUS, ESTIMATION METHOD, AND NON-TRANSITORY RECORDING MEDIUM

(71) Applicant: NEC Corporation, Tokyo (JP)

(72) Inventor: Akira HIROKAWA, Tokyo (JP)

(73) Assignee: NEC Corporation, Tokyo (JP)

(21) Appl. No.: 19/154,042

(22) PCT Filed: Jan. 17, 2024
(86) PCT No.: PCT/JP2024/001107

§ 371 Date: Aug. 6, 2025

(30) Foreign Application Priority Data
Feb. 14, 2023 (JP) 2023-020932

(51) Int. Cl. G06V 40/12; G06V 40/50

(52) U.S. Cl. CPC G06V 40/1365; G06V 40/50

(57) ABSTRACT

An estimation apparatus includes: an acquisition unit that acquires a rotated fingerprint image collected by rotating a finger; and an estimation unit that estimates a type of a finger included in the rotated fingerprint image, by inputting the acquired rotated fingerprint image into a learned model. According to such an estimation apparatus, it is possible to estimate the type of the finger included in the rotated fingerprint image with high accuracy.

### TECHNICAL FIELD

The present disclosure relates to technical fields of an estimation apparatus, an estimation method, and a recording medium.

### BACKGROUND ART

There is known an apparatus that performs various types of processing related to a fingerprint image. For example, Patent Literature 1 discloses a technique/technology for performing correction related to inclination of an image when comparing/matching a fingerprint image. Patent Literature 2 discloses a technique/technology for determining a type of a finger in a fingerprint image and detecting an error of the type of a fingerprinted finger. Patent Literature 3 discloses a technique/technology for identifying an abnormal area in fingerprints by using a learning model that is machine-learned.

### CITATION LIST

### Patent Literature

Patent Literature 1: Japanese Patent Publication No. 2001-076144

Patent Literature 2: Japanese Patent Publication No. 2016-053989

Patent Literature 3: Japanese Patent Publication No. 2018-165911

### SUMMARY

### Technical Problem

The present disclosure aims to improve the techniques/technologies disclosed in Citation List.

### Solution to Problem

An estimation apparatus according to an example aspect of the present disclosure includes: an acquisition unit that acquires a rotated fingerprint image collected by rotating a finger; and an estimation unit that estimates a type of a finger included in the rotated fingerprint image, by inputting the acquired rotated fingerprint image into a learned model.

An estimation method according to an example aspect of the present disclosure is an estimation method that is executed by at least one computer, the estimation method including: acquiring a rotated fingerprint image collected by rotating a finger; and estimating a type of a finger included in the rotated fingerprint image, by inputting the acquired rotated fingerprint image into a learned model.

A recording medium according to an example aspect of the present disclosure is a recording medium on which a computer program that allows at least one computer to execute an estimation method is recorded, the estimation method including: acquiring a rotated fingerprint image collected by rotating a finger; and estimating a type of a finger included in the rotated fingerprint image, by inputting the acquired rotated fingerprint image into a learned model.

### BRIEF DESCRIPTION OF DRAWINGS

FIG. 1 is a block diagram illustrating a hardware configuration of an estimation apparatus according to a first example embodiment.

FIG. 2 is a block diagram illustrating a functional configuration of the estimation apparatus according to the first example embodiment.

FIG. 3 is a flowchart illustrating a flow of operation of the estimation apparatus according to the first example embodiment.

FIG. 4 is a block diagram illustrating a functional configuration of an estimation apparatus according toa second example embodiment.

FIG. 5 is a flowchart illustrating a flow of operation of the estimation apparatus according to the second example embodiment.

FIG. 6 is a block diagram illustrating a functional configuration of an estimation apparatus according to a third example embodiment.

FIG. 7 is a flowchart illustrating a flow of operation of the estimation apparatus according to the third example embodiment.

FIG. 8 is a block diagram illustrating a functional configuration of an estimation apparatus according to a fourth example embodiment.

FIG. 9 is a flowchart illustrating a flow of operation of the estimation apparatus according to the fourth example embodiment.

FIG. 10 is a block diagram illustrating a functional configuration of an estimation apparatus according to the fifth example embodiment.

FIG. 11 is a flowchart illustrating a flow of operation of the estimation apparatus according to the fifth example embodiment.

FIG. 12 is a block diagram illustrating a functional configuration of an estimation apparatus according to a sixth example embodiment.

FIG. 13 is a flowchart illustrating a flow of operation of the estimation apparatus according to the sixth example embodiment.

FIG. 14 is a flowchart illustrating a flow of operation of an estimation apparatus according to a modified example of the sixth example embodiment.

FIG. 15 is a schematic configuration diagram illustrating a hardware configuration of an estimation apparatus according to a seventh example embodiment.

### DESCRIPTION OF EXAMPLE EMBODIMENTS

Hereinafter, an estimation apparatus, an estimation method, and a recording medium according to example embodiments will be described with reference to the drawings. cl First Example Embodiment

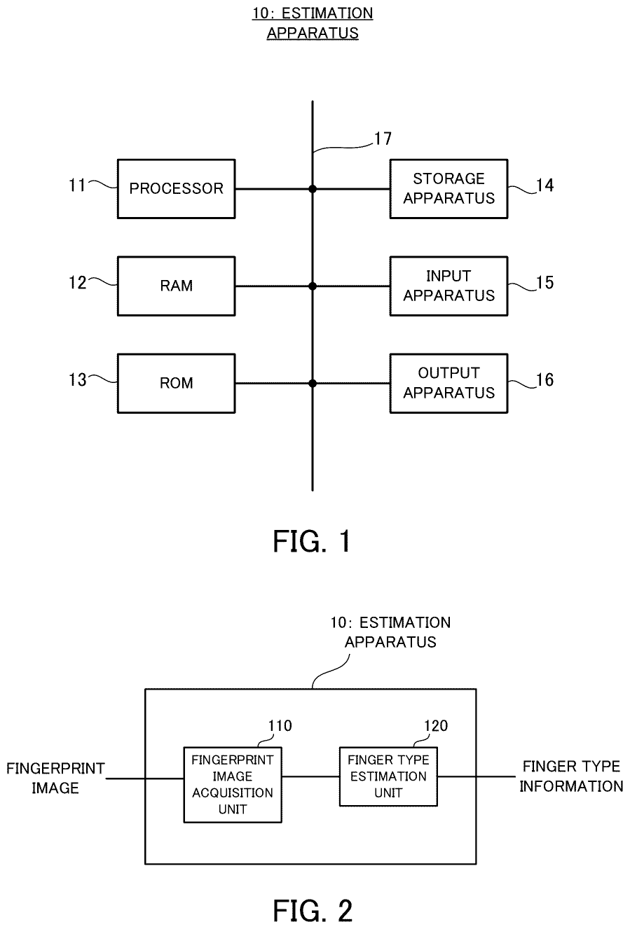

An estimation apparatus according to a first example embodiment will be described with reference to FIG. 1 to FIG. 3.

### (Hardware Configuration)

First, with reference to FIG. 1, a hardware configuration of the estimation apparatus according to the first example embodiment will be described. FIG. 1 is a block diagram illustrating the hardware configuration of the estimation apparatus according to the first example embodiment.

As illustrated in FIG. 1, an estimation apparatus 10 according to the first example embodiment includes a processor 11, a RAM (Random Access Memory) 12, and a ROM (Read Only Memory) 13. The estimation apparatus 10 may further include a storage apparatus 14, an input apparatus 15, and an output apparatus 16. The processor 11, the RAM 12, the ROM 13, the storage apparatus 14, the input apparatus 15, and the output apparatus 16 described above are connected via a data bus 17.

The processor 11 reads a computer program. For example, the processor 11 is configured to read a computer program stored in at least one of the RAM 12, the ROM 13, and the storage apparatus 14. Alternatively, the processor 11 may read a computer program stored on a computer-readable recording medium, by using a not-illustrated recording medium reading apparatus. The processor 11 may also acquire (i.e., read) a computer program from a not-illustrated apparatus disposed outside the estimation apparatus 10 via a network interface. The processor 11 controls the RAM 12, the storage apparatus 14, the input apparatus 15, and the output apparatus 16 by executing the read computer program. Especially in the present example embodiment, when the processor 11 executes the read computer program, a function block for acquiring a rotated fingerprint image and estimating a finger type is realized in the processor 11. That is, the processor 11 may function as a controller that exercises each control in the estimation apparatus 10.

The processor 11 may be configured as, for example, a CPU (Central Processing Unit), a GPU (Graphics Processing Unit), a FPGA (Field-Programmable Gate Array), a DSP (Digital Signal Processor), an ASIC (Application Specific Integrated Circuit), or a quantum processor. The processor 11 may be configured by using one of them, or a plurality of them in parallel.

The RAM 12 temporarily stores the computer program to be executed by processor 11. The RAM 12 temporarily stores data that are temporarily used by the processor 11 when the processor 11 is executing the computer program. The RAM 12 may be, for example, a D-RAM (Dynamic Random Access Memory) or a SRAM (Static Random Access Memory). In addition, another type of volatile memory may be used in place of the RAM 12.

The ROM 13 stores the computer program to be executed by the processor 11. The ROM 13 may also store other fixed data. The ROM 13 may be, for example, a P-ROM (Programmable Read Only Memory) or an EPROM (Erasable Read Only Memory). In addition, another type of nonvolatile memory may be used in place of the ROM 13.

The storage apparatus 14 stores data that are stored by the estimation apparatus 10 for a long time. The storage apparatus 14 may operate as a transitory storage apparatus of the processor 11. The storage apparatus 14 may include, for example, at least one of a hard disk apparatus, a magneto-optical disk apparatus, a SSD (Solid State Drive), and a disk array apparatus.

The input apparatus 15 is an apparatus that receives an input instruction from a user of the estimation apparatus 10. The input apparatus 15 may include, for example, at least one of a keyboard, a mouse, and a touch panel. The input apparatus 15 may be configured as a portable terminal such as a smartphone and a tablet. The input apparatus 15 may be an apparatus that allows audio input/voice input, including a microphone, for example.

The output apparatus 16 is an apparatus that outputs information about the estimation apparatus 10 to the outside. For example, the output apparatus 16 may be a display apparatus (e.g., a display) that is configured to display the information about the estimation apparatus 10. The output apparatus 16 may be configured as a portable terminal such as a smartphone and a tablet. The output apparatus 16 may also be an apparatus that outputs information in a format other than an image. For example, the output apparatus 16 may be a speaker that audio-outputs the information about the estimation apparatus 10.

A part of the hardware described in FIG. 1 may be provided as external apparatus of the estimation apparatus 10. For example, the estimation apparatus 10 may be configured to include only the processor 11, the RAM 12, and the ROM 13 described above. The other components (i.e., the storage apparatus 14, the input apparatus 15, and the output apparatus 16) may be provided in an external apparatus connected to the estimation apparatus 10. In addition, the estimation apparatus 10 may realize a part of an arithmetic function by using an external apparatus (e.g., an external server or cloud, etc.).

### (Functional Configuration)

Next, with reference to FIG. 2, a functional configuration of the estimation apparatus 10 according to the first example embodiment will be described. FIG. 2 is a block diagram illustrating the functional configuration of the estimation apparatus according to the first example embodiment.

As illustrated in FIG. 2, the estimation apparatus 10 according to the first example embodiment includes, as components for realizing the functions thereof, a fingerprint image acquisition unit 110 and a finger type estimation unit 120. Each of the fingerprint image acquisition unit 110 and the finger type estimation unit 120 may be a processing block realized, for example, by the processor 11 described above (see FIG. 1).

The fingerprint image acquisition unit 110 is configured to acquire a rotated fingerprint image. The rotated fingerprint image is a fingerprint image collected by rotating a finger, and includes not only a fingerprint on the pad of the finger, but also a fingerprint on the side of the finger. The fingerprint image acquisition unit 110 may directly acquire a rotated fingerprint image collected by using a scanner or the like, or may acquire a rotated fingerprint image stored in a database or the like (i.e., a rotated fingerprint image collected in the past). The rotated fingerprint image acquired by the fingerprint image acquisition unit 110 is outputted to the finger type estimation unit 120.

The finger type estimation unit 120 is configured to estimate a type of a finger (hereinafter referred to as a “finger type”) included in the rotated fingerprint image acquired by the fingerprint image acquisition unit 110. The finger type estimation unit 120 estimates the finger type by inputting the rotated fingerprint image into a model that is already learned/trained (hereinafter referred to as a “learning model”). The learning model is a model that is learned/trained in advance by using training data, and when a rotated fingerprint image is inputted, the learning model outputs the finger type of the rotated fingerprint image. The learning model may be a model that is machine-learned by inputting, for example, pair information on a pair of the fingerprint image and the finger type, which is training data. The learning model may be a neural network that is learned/trained by deep learning, for example.

When the learning model is learned/trained, it is desirable to prepare as much training data as possible in order to improve accuracy of finger type estimation. However, in a case where there is only a small number of pieces of training data that can be prepared, the number of pieces of training data may be increased, for example, by generating similar data to the training data, or adding perturbations to the training data. In this case, the training data may be generated, for example, by using a GAN (generative adversarial network).

### (Flow of Operation)

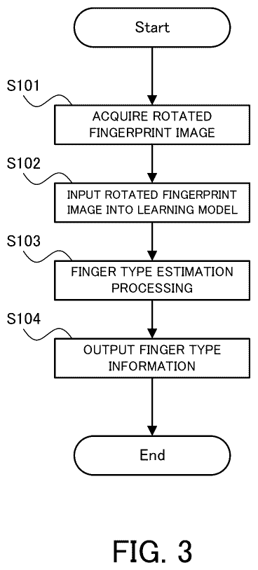

Next, with reference to FIG. 3, a flow of operation of the estimation apparatus 10 according to the first example embodiment will be described. FIG. 3 is a flowchart illustrating the flow of the operation of the estimation apparatus according to the first example embodiment.

As illustrated in FIG. 3, when the operation of the estimation apparatus 10 according to the first example embodiment is started, first, the fingerprint image acquisition unit 110 acquires the rotated fingerprint image (step S101). Then, the finger type estimation unit 120 inputs the rotated fingerprint image acquired by the fingerprint image acquisition unit 110 into the learning model (step S102).

Then, based on the rotated fingerprint image inputted into the learning model, the type of the finger included in the rotated fingerprint image is estimated (step S103). Then, the finger type estimation unit 120 outputs finger type information indicating the type of the finger included in the rotated fingerprint image, based on an output of the learning model (step S104). The finger type information outputted by the finger type estimation unit 120 may be stored (registered) in association with the rotated fingerprint image, for example. This registration processing using the finger type information will be described in detail in another example embodiment later.

### (Technical Effect)

Next, a technical effect obtained by the estimation apparatus 10 according to the first example embodiment will be described.

As described in FIG. 1 to FIG. 3, in the estimation apparatus 10 according to the first example embodiment, the type of the finger included in the rotated fingerprint image is estimated by inputting the rotated fingerprint image into the learning model. The finger type estimation based on the rotated fingerprint image alone is not easy even for forensic experts specializing in fingerprints. However, according to the estimation apparatus 10 in the present example embodiment, it is possible to estimate the type of the finger included in the rotated fingerprint image with high accuracy.

### Second Example Embodiment

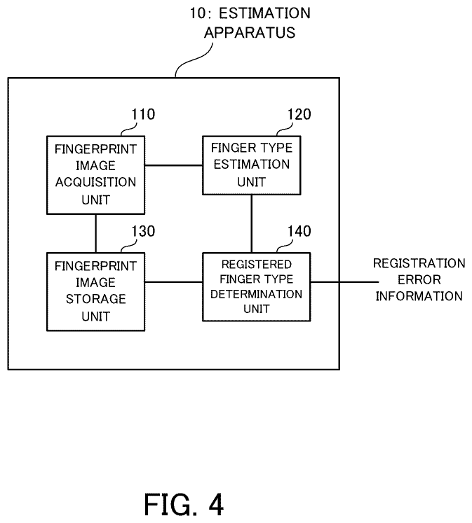

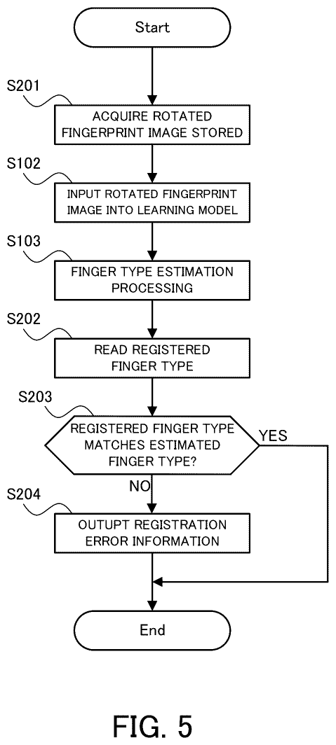

The estimation apparatus 10 according to a second example embodiment will be described with reference to FIG. 4 and FIG. 5. The second example embodiment partially differs from the first example embodiment described above only in its configuration and operation, and may be the same as the first example embodiment in the other parts. For this reason, a part differing from the first example embodiment already described will be described in detail below, and a description of the other overlapping parts will be omitted as appropriate.

### (Functional Configuration)

First, with reference to FIG. 4, a functional configuration of the estimation apparatus 10 according to the second example embodiment will be described. FIG. 4 is a block diagram illustrating the functional configuration of the estimation apparatus according to the second example embodiment. In FIG. 4, the same components as those illustrated in FIG. 2 carry the same reference numerals.

As illustrated in FIG. 4, the estimation apparatus 10 according to the second example embodiment includes, as components for realizing the functions thereof, the fingerprint image acquisition unit 110, the finger type estimation unit 120, a fingerprint image storage unit 130, and a registered finger type determination unit 140. That is, the estimation apparatus 10 according to the second example embodiment further includes the fingerprint image storage unit 130 and the registered finger type determination unit 140, in addition to the configuration in the first example embodiment (see FIG. 2). Each of the fingerprint image storage unit 130 and the registered finger type determination unit 140 may be realized by, for example, the processor 11 or the storage apparatus 14 described above (see FIG. 1).

The fingerprint image storage unit 130 is configured to store the rotated fingerprint image in association with the finger type. For example, the fingerprint image storage unit 130 stores a rotated fingerprint image collected from a thumb, with information indicating a “thumb” added thereto. The fingerprint image storage unit 130 may be configured to store rotated fingerprint images of a plurality of fingers, in association with the finger types. For example, the fingerprint image storage unit 130 may be configured to store rotated fingerprint images of all ten fingers of both hands, in association with the respective finger types. Additionally, the fingerprint image storage unit 130 may be configured to store a planar fingerprint image (i.e., a fingerprint image of the pad of the finger collected without rotating the finger), in addition to the rotated fingerprint image. In this case, the planar fingerprint image may also be stored in association with the finger type. Furthermore, the fingerprint image storage unit 130 may be configured to store the rotated fingerprint image for each user. For example, the fingerprint image storage unit 130 may be configured to store 10 rotated fingerprint images of both hands of a user A, 10 rotated fingerprint images of both hands of a user B, and 10 rotated fingerprint images of both hands of a user C. The rotated fingerprint image stored in the fingerprint image storage unit 130 is configured to be read as appropriate by the registered finger type determination unit 140.

The fingerprint image storage unit 130 may be configured as a database for storing the fingerprint image used in a fingerprint matching/identification system, for example. More specifically, the fingerprint image storage unit 130 may be configured as a database for a fingerprint matching/identification system managed by the police.

The registered finger type determination unit 140 is configured to determine whether or not the finger type stored in the fingerprint image storage unit 130 (i.e., the type of the finger associated with the rotated fingerprint image) matches the finger type estimated by the finger type estimation unit 120 (i.e., the type of the finger estimated by inputting the rotated fingerprint image into the learning model). Furthermore, the registered finger type determination unit 140 is configured to output information indicating that the finger type stored in the fingerprint image storage unit 130 is incorrect, when the finger type stored in the fingerprint image storage unit 130 does not match the finger type estimated by the finger type estimation unit 120.

### (Flow of Operation)

Next, with reference to FIG. 5, a flow of operation of the estimation apparatus 10 according to the second example embodiment will be described. FIG. 5 is a flowchart illustrating the flow of the operation of the estimation apparatus according to the second example embodiment. In FIG. 5, the same steps as those described in FIG. 3 carry the same reference numerals.

As illustrated in FIG. 5, when the operation of the estimation apparatus 10 according to the second example embodiment is started, first, the fingerprint image acquisition unit 110 acquires the rotated fingerprint image stored in the fingerprint image storage unit 130 (step S201).

Then, the finger type estimation unit 120 inputs the rotated fingerprint image acquired by the fingerprint image acquisition unit 110 into the learning model (step S102). Then, based on the rotated fingerprint image inputted into the learning model, the type of the finger included in the rotated fingerprint image is estimated (step S103).

Then, the registered finger type determination unit 140 reads the finger type stored in the fingerprint image storage unit 130 (step S202). That is, it reads information indicating the type of the finger associated with the rotated fingerprint image acquired in step S201. Then, the registered finger type determination unit 140 determines whether or not the finger type stored in the fingerprint image storage unit 130 matches the finger type estimated by the finger type estimation unit 120 (step S203).

When the finger type stored in the fingerprint image storage unit 130 does not match the finger type estimated by the finger type estimation unit 120 (the step S203: NO), the registered finger type determination unit 140 outputs information indicating that the finger type stored in the fingerprint image storage unit 130 is incorrect (hereinafter referred to as “registration error information”) (step S204).

The registered finger type determination unit 140 may display the registration error information to a user (administrator) of the apparatus via a display or the like. For example, the registered finger type determination unit 140 may display each of the rotated fingerprint image in which the finger type is incorrectly stored, the already stored finger type (i.e., an incorrect finger type), and the estimated finger type (i.e., a correct finger type). In addition, the registered finger type determination unit 140 may perform processing of correcting or deleting the finger type, for the rotated fingerprint image in which the finger type is incorrectly stored. Alternatively, the registered finger type determination unit 140 may output a message prompting re-registration of the rotated fingerprint image in which the finger type is incorrectly stored. For example, the registered finger type determination unit 140 may output a message “This rotated fingerprint image is registered with an incorrect finger type. Please re-register with the correct finger type.”

On the other hand, when the finger type stored in the fingerprint image storage unit 130 matches the finger type estimated by the finger type estimation unit 120 (the step S203: YES), the step S204 is omitted. That is, the registered finger type determination unit 140 does not output the registration error information. In this case, the registered finger type determination unit 140 may output information indicating that the finger type stored in the fingerprint image storage unit 130 is correct.

In a case where a plurality of rotated fingerprint images are stored in the fingerprint image storage unit 130, the above-described series of processing steps may be repeatedly performed on each of the plurality of rotated fingerprint images. That is, the estimation apparatus 10 according to the present example embodiment may determine whether or not the stored finger types are correct for all of the plurality of rotated fingerprint images stored in the fingerprint image storage unit 130.

### (Technical Effect)

Next, a technical effect obtained by the estimation apparatus 10 according to the second example embodiment will be described.

As described in FIG. 4 and FIG. 5, in the estimation apparatus 10 according to the second example embodiment, it is determined whether or not the finger type in the stored rotated fingerprint image matches the finger type estimated from the rotated fingerprint image. In this way, it is possible to appropriately determine an error of the finger type of the rotated fingerprint image stored.

### Third Example Embodiment

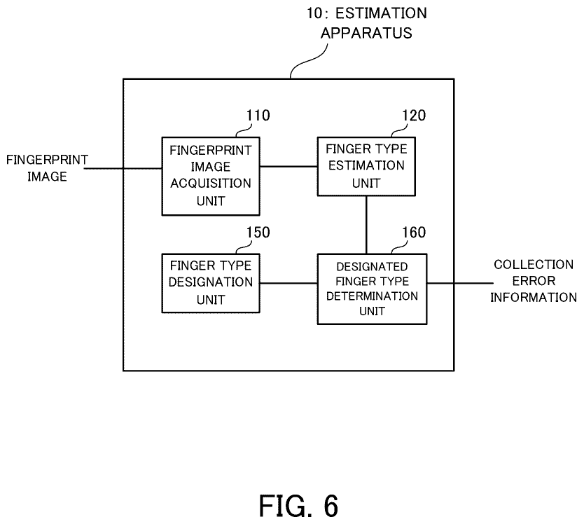

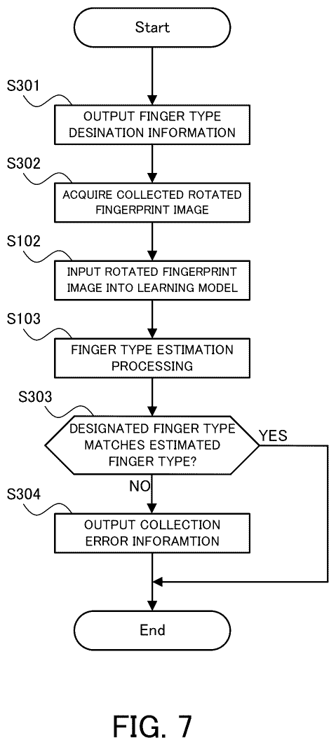

The estimation apparatus 10 according to a third example embodiment will be described with reference to FIG. 6 and FIG. 7. The third example embodiment partially differs from the first and second example embodiments described above only in its configuration and operation, and may be the same as the first and second example embodiments in the other parts. For this reason, a part differing from each of the example embodiments described above will be described in detail below, and a description of the other overlapping parts will be omitted as appropriate.

### (Functional Configuration)

First, with reference to FIG. 6, a functional configuration of the estimation apparatus 10 according to the third example embodiment will be described. FIG. 6 is a block diagram illustrating the functional configuration of the estimation apparatus according to the third example embodiment. In FIG. 6, the same components as those described in FIG. 2 carry the same reference numerals.

As illustrated in FIG. 6, the estimation apparatus 10 according to the third example embodiment includes, as components for realizing the functions thereof, the fingerprint image acquisition unit 110, the finger type estimation unit 120, a finger type designation unit 150, and a designated finger type determination unit 160. That is, the estimation apparatus 10 according to the third example embodiment further includes the finger type designation unit 150 and the designated finger type determination unit 160, in addition to the configuration in the first example embodiment (see FIG. 2). Each of the finger type designation unit 150 and the designated finger type determination unit 160 may be a processing block realized, for example, by the processor 11 described above.

The finger type designation unit 150 is configured to designate the type of the finger from which the rotated fingerprint image is to be collected. For example, the finger type designation unit 150 may be configured to designate a predetermined type of the finger. Alternatively, the finger type designation unit 150 may be configured to sequentially designate the type of the finger from which the rotated fingerprint image is to be collected, in accordance with a predetermined acquisition order. The finger type designation unit 150 may output information indicating the designated finger type, to a target from whom the rotated fingerprint image is to be collected. For example, the finger type designation unit 150 may display a message such as “Please scan the fingerprint of your right index finger” on a display.

The designated finger type determination unit 160 is configured to determine whether or not the finger type designated by the finger type designation unit 150 matches the finger type estimated by the finger type estimation unit 120 using the collected rotated fingerprint image. Furthermore, the designated finger type determination unit 160 is configured to output information indicating that the type of the finger from which the rotated fingerprint image is collected, is incorrect, when the finger type designated by the finger type designation unit 150 does not match the finger type estimated by the finger type estimation unit 120.

### (Flow of Operation)

Next, with reference to FIG. 7, a flow of operation of the estimation apparatus 10 according to the third example embodiment will be described. FIG. 7 is a flowchart illustrating the flow of the operation of the estimation apparatus according to the third example embodiment. In FIG. 7, the same steps as those described in FIG. 3 will carry the same reference numerals.

As illustrated in FIG. 7, when the operation of the estimation apparatus 10 according to the third example embodiment is started, first, the finger type designation unit 150 outputs information for designating the finger type for which the rotated fingerprint image is to be collected (step S301). This causes the target to perform an action of scanning the designated finger. Then, the fingerprint image acquisition unit 110 acquires the rotated fingerprint image acquired from the target (step S302).

Then, the finger type estimation unit 120 inputs the rotated fingerprint image acquired by the fingerprint image acquisition unit 110 into the learning model (step S102). Then, based on the rotated fingerprint image inputted into the learning model, the type of the finger included in the rotated fingerprint image is estimated (step S103).

Then, the designated finger type determination unit 160 determines whether or not the finger type designated by the finger type designation unit 150 matches the finger type estimated by the finger type estimation unit 120 (step S303). That is, the designated finger type determination unit 160 determines whether or not the type of the finger designated to be collected matches the type of the finger actually collected.

When the finger type designated by the finger type designation unit 150 does not match the finger type estimated by the finger type estimation unit 120 (the step S303: NO), the designated finger type determination unit 160 outputs information indicating that the finger type for which the rotated fingerprint image is collected, is incorrect (hereinafter referred to as “collection error information”) (step S304).

The designated finger type determination unit 160 may display the collection error information to the target (the user from whom the rotated fingerprint image is collected) via a display or the like. For example, the designated finger type determination unit 160 may display each of the collected rotated fingerprint image, the finger type designated by the finger type designation unit 150, and the finger type actually collected (i.e., the type of the finger that is incorrectly collected). In addition, the designated finger type determination unit 160 may output a message prompting re-collection of the rotated fingerprint image of the designated finger type. For example, the designated finger type determination unit 160 may output a message such as “It appears that the fingerprint of your right middle finger is scanned incorrectly. Please scan the fingerprint of your right index finger.”

On the other hand, when the finger type designated by the finger type designation unit 150 matches the finger type estimated by the finger type estimation unit 120 (the step S303: YES), the step S304 is omitted. That is, the designated finger type determination unit 160 does not output the collection error information. In this case, the designated finger type determination unit 160 may output information indicating that the type of the finger from which the rotated fingerprint image is collected, is correct.

### (Technical Effect)

Next, a technical effect obtained by the estimation apparatus 10 according to the third example embodiment will be described.

As described in FIG. 6 and FIG. 7, in the estimation apparatus 10 according to the third example embodiment, it is determined whether or not the type of the finger from which the rotated fingerprint image is collected, matches the designated finger type. In this way, it is possible to appropriately detect an error of the finger from which rotated fingerprint image is collected. Therefore, it is possible to collect the rotated fingerprint image of the designated finger without an error. In addition, in a case where the order of the fingers to be collected is designated, the rotated fingerprint images may be captured in the designated order. As a result, it is possible to prevent the collected rotated fingerprint image from being registered as that of a different finger.

### Fourth Example Embodiment

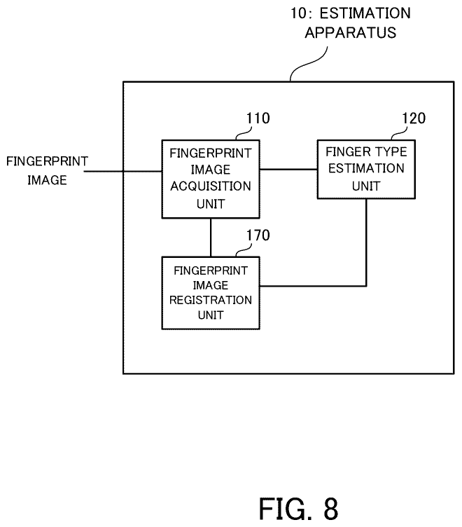

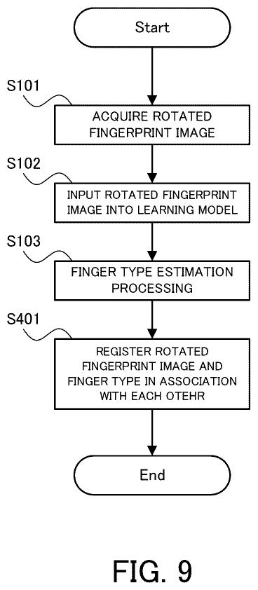

The estimation apparatus 10 according toa fourth example embodiment will be described with reference to FIG. 8 and FIG. 9. The fourth example embodiment partially differs from the first to third example embodiments described above only in its configuration and operation, and may be the same as the first to third example embodiments in the other parts. For this reason, a part differing from each of the example embodiments described above will be described in detail below, and a description of the other overlapping parts will be omitted as appropriate.

### (Functional Configuration)

First, with reference to FIG. 8, a functional configuration of the estimation apparatus 10 according to the fourth example embodiment will be described. FIG. 8 is a block diagram illustrating the functional configuration of the estimation apparatus according to the fourth example embodiment. In FIG. 8, the same components as those described in FIG. 2 carry the same reference numerals.

As illustrated in FIG. 8, the estimation apparatus 10 according to the fourth example embodiment includes, as components for realizing the functions thereof, the fingerprint image acquisition unit 110, the finger type estimation unit 120, and a fingerprint image registration unit 170. That is, the estimation apparatus 10 according to the fourth example embodiment further includes the fingerprint image registration unit 170, in addition to the configuration in the first example embodiment (see FIG. 2). The fingerprint image registration unit 170 may be realized, for example, by the processor 11 and the storage apparatus 14 described above (see FIG. 1).

The fingerprint image registration unit 170 is configured to register the rotated fingerprint image in association with the finger type of the rotated fingerprint image. That is, the fingerprint image registration unit 170 is configured to register the rotated fingerprint image acquired by the fingerprint image acquisition unit 110 and the finger type estimated from the rotated fingerprint image, in association with each other. The fingerprint image registration unit 170 may be configured to store (register) the rotated fingerprint image and finger type in the fingerprint image storage unit 130 described in the second example embodiment (see FIG. 4), for example.

### (Flow of Operation)

Next, with reference to FIG. 9, a flow of operation of the estimation apparatus 10 according to the fourth example embodiment will be described. FIG. 9 is a flowchart illustrating the flow of the operation of the estimation apparatus according to the fourth example embodiment. In FIG. 9, the same steps as those described in FIG. 3 carry the same reference numerals.

As illustrated in FIG. 9, when the operation of the estimation apparatus 10 according to the fourth example embodiment is started, first, the fingerprint image acquisition unit 110 acquires the rotated fingerprint image (step S101). For example, the fingerprint image acquisition unit 110 acquires the rotated fingerprint image collected from the target by using a scanner or the like.

Then, the finger type estimation unit 120 inputs the rotated fingerprint image acquired by the fingerprint image acquisition unit 110 into the learning model (step S102). Then, based on the rotated fingerprint image inputted into the learning model, the type of the finger included in the rotated fingerprint image is estimated (step S103).

Then, the fingerprint image registration unit 170 registers the rotated fingerprint image acquired by the fingerprint image acquisition unit 110 and the finger type estimated by the finger type estimation unit 120, in association with each other (step S401). For example, in a case where the acquired rotated fingerprint image is estimated to be from a right index finger, the fingerprint image registration unit 170 registers the acquired rotated fingerprint image and information indicating that the rotated fingerprint image is collected from a right index finger, in association with each other.

### (Technical Effect)

Next, a technical effect obtained by the estimation apparatus 10 according to the fourth example embodiment will be described.

As described in FIG. 8 and FIG. 9, in the estimation apparatus 10 according to the fourth example embodiment, the rotated fingerprint image and the type of the finger of the rotated fingerprint image are registered in association with each other. In the present example embodiment, since the finger type estimation unit 120 estimates the finger type of the rotated fingerprint image with high accuracy, it is possible to prevent/control that the rotated fingerprint image is registered with the incorrect finger type. In addition, since the finger type of the rotated fingerprint image collected is automatically estimated, it is possible to appropriately register the rotated fingerprint image without designating the order of the fingers to be collected.

### Fifth Example Embodiment

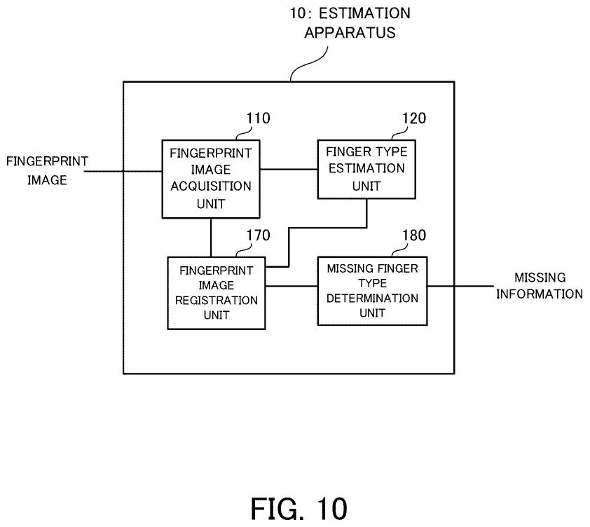

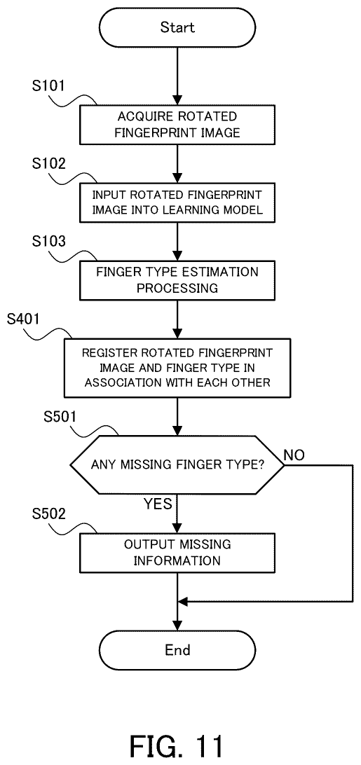

The estimation apparatus 10 according to a fifth example embodiment will be described with reference to FIG. 10 and FIG. 11. The fifth example embodiment partially differs from the fourth example embodiment described above only in its configuration and operation, and may be the same as the fourth example embodiment in the other parts. For this reason, a part differing from each of the example embodiments described above will be described in detail below, and a description of the other overlapping parts will be omitted as appropriate.

### (Functional Configuration)

First, with reference to FIG. 10, a functional configuration of the estimation apparatus 10 according to the fifth example embodiment will be described. FIG. 10 is a block diagram illustrating the functional configuration of the estimation apparatus according to the fifth example embodiment. In FIG. 10, the same components as those described in FIG. 8 carry the same reference numerals.

As illustrated in FIG. 10, the estimation apparatus 10 according to the fifth example embodiment includes, as components for realizing the functions thereof, the fingerprint image acquisition unit 110, the finger type estimation unit 120, the fingerprint image registration unit 170, and a missing finger type determination unit 180. That is, the estimation apparatus 10 according to the fifth example embodiment further includes the missing finger type determination unit 180, in addition to the configuration in the fourth example embodiment (see FIG. 8). The missing finger type determination unit 180 may be a processing block realized, for example, by the processor 11 described above (see FIG. 1).

The missing finger type determination unit 180 is configured to determine whether or not there is a shortage of the rotated fingerprint image registered in the fingerprint image registration unit 170. For example, the missing finger type determination unit 180 may be configured to determine whether or not the rotated fingerprint images are registered for all ten fingers of both hands. The missing finger type determination unit 180 is configured to output information prompting collection of the rotated fingerprint image of a missing finger (hereinafter referred to as “missing information”), when there is a shortage of the rotated fingerprint image registered. The missing information may include information indicating the type of a missing finger, for example. The missing information may also include information indicating the type of the finger already collected.

### (Flow of Operation)

Next, with reference to FIG. 11, a flow of operation of the estimation apparatus 10 according to the fifth example embodiment will be described. FIG. 11 is a flowchart illustrating the flow of the operation of the estimation apparatus according to the fifth example embodiment. In FIG. 11, the same steps as those described in FIG. 9 carry the same reference numerals.

As illustrated in FIG. 11, when the operation of the estimation apparatus 10 according to the fifth example embodiment starts, first, the fingerprint image acquisition unit 110 acquires the rotated fingerprint image (step S101). Then, the finger type estimation unit 120 inputs the rotated fingerprint image acquired by the fingerprint image acquisition unit 110 into the learning model (step S102).

Then, based on the rotated fingerprint image inputted into the learning model, the type of the finger included in the rotated fingerprint image is estimated (step S103). Then, the fingerprint image registration unit 170 registers the rotated fingerprint image acquired by the fingerprint image acquisition unit 110 and the finger type estimated by the finger type estimation unit 120, in association with each other (step S401).

Then, the missing finger type determination unit 180 determines whether or not there is a shortage of the rotated fingerprint image registered by the fingerprint image registration unit 170 (step S501). Here, an example is given in which the processing of determining whether or not there is a shortage of the rotated fingerprint image (i.e., the processing in the step S501) is performed immediately after the processing of registering the rotated fingerprint image (i.e., the processing in the step S401); however, the step S501 may be performed a certain time after the completion of the processing in the step S401. For example, the missing finger type determination unit 180 may be configured to determine whether or not there is a shortage of past rotated fingerprint images registered several months or several years ago.

When there is a shortage of the registered rotated fingerprint image (the step S501: YES), the missing finger type determination unit 180 outputs the missing information (step S502). For example, in a case where the rotated fingerprint image of a right index finger is missing, the missing finger type determination unit 180 may output a message such as “The fingerprint of your right index finger is missing. Please acquire the fingerprint of the right index finger.”

On the other hand, when there is no shortage of the registered rotated fingerprint image (the step S501: NO), the above-mentioned step S502 is omitted. That is, the missing finger type determination unit 180 does not output the missing information. In this case, the missing finger type determination unit 180 may output information indicating that all the rotated fingerprint images are collected.

### (Technical Effect)

Next, a technical effect obtained by the estimation apparatus 10 according to the fifth example embodiment will be described.

As described in FIG. 10 and FIG. 11, in the estimation apparatus 10 according to the fifth example embodiment, when there is a missing finger type in the registered rotated fingerprint image, information prompting collection of the rotated fingerprint image of the missing finger types is outputted. In this way, it is possible to appropriately detect the missing finger type in the registered rotated fingerprint image. Furthermore, since it is possible to prompt collection of the rotated fingerprint image of the missing finger, it is possible to quickly supplement the missing rotated fingerprint image.

### Sixth Example Embodiment

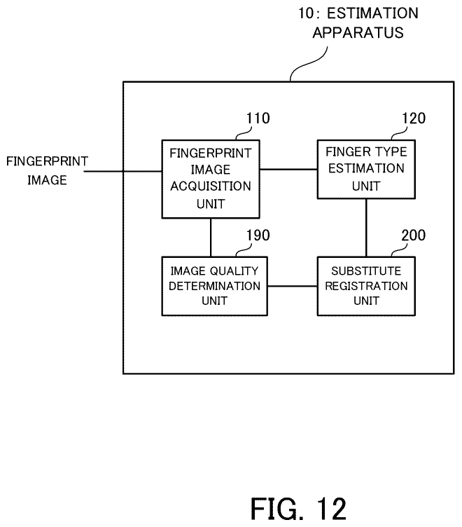

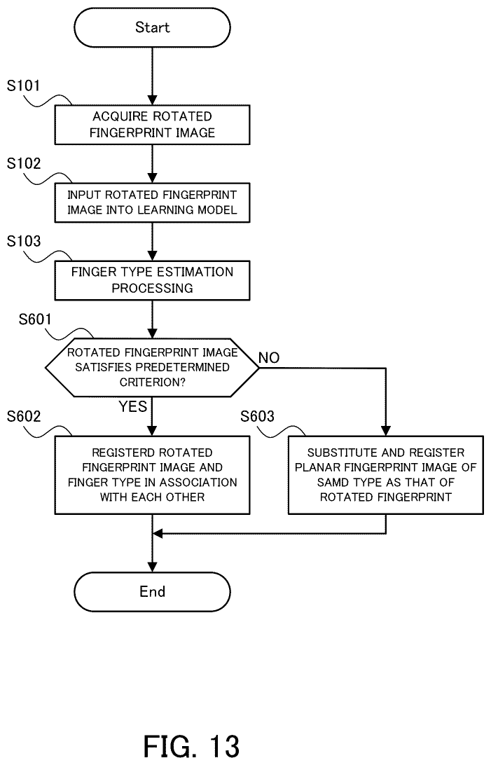

The estimation apparatus 10 according to a sixth example embodiment will be described with reference to FIG. 12 and FIG. 13. The sixth example embodiment partially differs from the first to fifth example embodiments described above only in its configuration and operation, and may be the same as the first to fifth example embodiments in the other parts. For this reason, a part differing from each of the example embodiments described above will be described in detail below, and a description of the other overlapping parts will be omitted as appropriate.

### (Functional Configuration)

First, with reference to FIG. 12, a functional configuration of the estimation apparatus 10 according to the sixth example embodiment will be described. FIG. 12 is a block diagram illustrating the functional configuration of the estimation apparatus according to the sixth example embodiment. In FIG. 12, the same components as those described in FIG. 2 carry the same reference numerals.

As illustrated in FIG. 12, the estimation apparatus 10 according to the sixth example embodiment includes, as components for realizing the functions thereof, the fingerprint image acquisition unit 110, the finger type estimation unit 120, an image quality determination unit 190, and a substitute registration unit 200. That is, the estimation apparatus 10 according to the sixth example embodiment further includes the image quality determination unit 190 and the substitute registration unit 200, in addition to the configuration in the first example embodiment (see FIG. 2). Each of the image quality determination unit 190 and the substitute registration unit 200 may be realized by, for example, the processor 11 or the storage apparatus 14 described above (see FIG. 1).

The image quality determination unit 190 is configured to determine quality of the rotated fingerprint image acquired by the fingerprint image acquisition unit 110. Specifically, the image quality determination unit 190 is configured to determine whether or not the quality of the rotated fingerprint image satisfies a predetermined criterion. The “predetermined criterion” here is set in advance as a criterion for determining whether or not the quality of the rotated fingerprint image is sufficient for its operational use. For example, the predetermined criterion may be set as a criterion for determining whether or not the quality of the rotated fingerprint image is suitable for fingerprint matching.

The substitute registration unit 200 is configured to register a planar fingerprint image of the same type as that of the rotated fingerprint image, instead of the rotated fingerprint image, when it is determined that the quality of the rotated fingerprint image does not satisfy the predetermined criterion. For example, when the quality of the rotated fingerprint image of a right index finger does not satisfy the predetermined criterion, the substitute registration unit 200 registers the planar fingerprint image of the right index finger in association with information indicating that the finger type included in the image is a right index finger. The planar fingerprint image registered instead of the rotated fingerprint image may be an image that is already registered. That is, the planar fingerprint image may be an image that is collected separately from the rotated fingerprint image and that is already registered.

In addition, the substitute registration unit 200 may register the rotated fingerprint image in association with information indicating the finger type, when it is determined that the quality of the rotated fingerprint image satisfies the predetermined criterion. That is, the substitute registration unit 200 may have the same function as that of the fingerprint image registration unit 170 described in the fourth example embodiment (see FIG. 8).

### (Flow of Operation)

Next, with reference to FIG. 13, a flow of operation of the estimation apparatus 10 according to the sixth example embodiment will be described. FIG. 13 is a flowchart illustrating the flow of the operation of the estimation apparatus according to the sixth example embodiment. In FIG. 13, the same steps as those described in FIG. 3 carry the same reference numerals.

As illustrated in FIG. 13, when the operation of the estimation apparatus 10 according to the sixth example embodiment is started, first, the fingerprint image acquisition unit 110 acquires the rotated fingerprint image (step S101). Then, the finger type estimation unit 120 inputs the rotated fingerprint image acquired by the fingerprint image acquisition unit 110 into the learning model (step S102). Then, based on the rotated fingerprint image inputted into the learning model, the type of the finger included in the rotated fingerprint image is estimated (step S103).

Then, the image quality determination unit 190 determines whether or not the quality of the rotated fingerprint image acquired by the fingerprint image acquisition unit 110 satisfies the predetermined criterion (step S601). The processing of determining the quality of the rotated fingerprint image may be performed immediately after the rotated fingerprint image is acquired. That is, the step S601 may be performed simultaneously in parallel with the steps S102 and S103, or may be performed before or after them.

When the quality of the rotated fingerprint image satisfies the predetermined criterion (the step S601: YES), the substitute registration unit 200 registers the rotated fingerprint image in association with information indicating the estimated finger type (step S602). On the other hand, when the quality of the rotated fingerprint image does not satisfy the predetermined criterion (the step S601: NO), the substitute registration unit 200 registers the planar fingerprint image of the same finger type as that of the rotated fingerprint image, instead of the rotated fingerprint image (step S603).

### (Technical Effect)

Next, a technical effect obtained by the estimation apparatus 10 according to the sixth example embodiment will be described.

As described in FIG. 12 and FIG. 13, in the estimation apparatus 10 according to the sixth example embodiment, when the quality of the rotated fingerprint image does not satisfy the predetermined criterion, the planar fingerprint image is registered instead of the rotated fingerprint image. In this way, it is possible to prevent the registration of a low-quality rotated fingerprint image that is unsuitable for operation. In addition, it is possible to prevent the finger type from being omitted, by registering the planar fingerprint image instead of the rotated fingerprint image.

### Modified Example

The processing of substituting and registering the planar fingerprint image described in the sixth example embodiment (i.e., the processing in the step S603 in FIG. 13) may be performed when it is determined that the finger type is incorrect. For example, the substitute registration unit 200 may be configured to perform the processing of registering the planar fingerprint image instead of the already stored rotated fingerprint image, when the registration finger type determination unit 140 described in the second example embodiment determines that the finger type stored in the fingerprint image storage unit 130 does not match the finger type estimated by the finger type estimation unit 120. Hereinafter, a flow of operation in this modified example will be specifically described.

### (Flow of Operation)

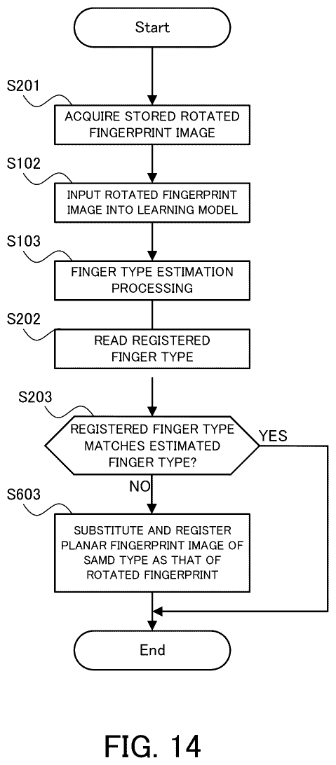

With reference to FIG. 14, the flow of the operation of the estimation apparatus 10 according to the modified example of the sixth example embodiment will be described. FIG. 14 is a flowchart illustrating the operation of the estimation apparatus according to the modified example of the sixth example embodiment. In FIG. 14, the same steps as those described in FIG. 5 and FIG. 13 carry the same reference numerals.

As illustrated in FIG. 14, when the operation of the estimation apparatus 10 according to the modified example of the sixth example embodiment is started, first, the fingerprint image acquisition unit 110 acquires the rotated fingerprint image stored in the fingerprint image storage unit 130 (step S201).

Then, the finger type estimation unit 120 inputs the rotated fingerprint image acquired by the fingerprint image acquisition unit 110 into the learning model (step S102). Then, based on the rotated fingerprint image inputted into the learning model, the type of the finger included in the rotated fingerprint image is estimated (step S103).

Then, the registered finger type determination unit 140 reads the finger type stored in the fingerprint image storage unit 130 (step S202). Then, the registered finger type determination unit 140 determines whether or not the finger type stored in the fingerprint image storage unit 130 matches the finger type estimated by the finger type estimation unit 120 (step S203).

When the finger type stored in the fingerprint image storage unit 130 does not match the finger type estimated by the finger type estimation unit 120 (the step S203: NO), the substitute registration unit 200 registers the planar fingerprint image of the same finger type as that of the rotated fingerprint image, instead of the rotated fingerprint image (step S603). That is, the substitute registration unit 200 overwrites the rotated fingerprint image already stored in the fingerprint image storage unit 130 (the rotated fingerprint image registered with the incorrect finger type), with the planar fingerprint image of the correct finger type.

On the other hand, when the finger type stored in the fingerprint image storage unit 130 matches the finger type estimated by the finger type estimation unit 120 (the step S203: YES), the step S603 is omitted. That is, the substitute registration unit 200 does not perform the processing of registering the planar fingerprint image instead of the rotated fingerprint image.

### (Technical Effect)

Next, a technical effect obtained by the estimation apparatus 10 according to the modified example of the sixth example embodiment will be described.

As described in FIG. 14, in the estimation apparatus 10 according to the modified example in the sixth example embodiment, in a case where the registered finger type is incorrect, the planar fingerprint image is registered instead of the rotated fingerprint image. In this way, even when the rotated fingerprint image of the incorrect finger type is registered, it is possible to correct an error of the finger type by replacing it with the planar fingerprint image of the correct finger type.

### Seventh Example Embodiment

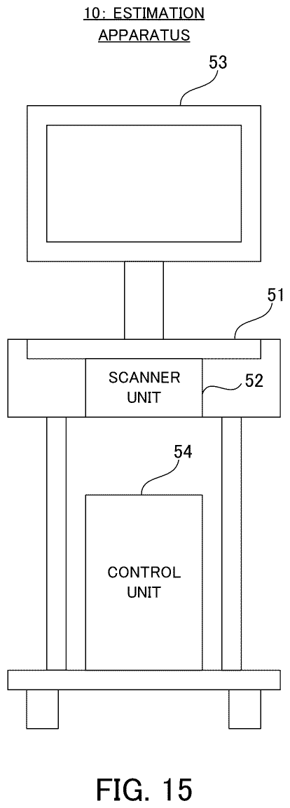

The estimation apparatus 10 according to a seventh example embodiment will be described with reference to FIG. 15. The seventh example embodiment is an example embodiment that describes a more specific hardware configuration of the estimation apparatus 10 described in the first to sixth example embodiments above, and may be the same as the first to sixth example embodiments in its functional configuration and flow of operation. For this reason, a part differing from each of the example embodiments described above will be described in detail below, and a description of the other overlapping parts will be omitted as appropriate.

### (Hardware Configuration)

First, with reference to FIG. 15, a hardware configuration of the estimation apparatus 10 according to the seventh example embodiment will be described. FIG. 15 is a schematic configuration diagram illustrating the hardware configuration of the estimation apparatus according to the seventh example embodiment.

As illustrated in FIG. 15, the estimation apparatus 10 according to the seventh example embodiment includes a touch panel 51, a scanner unit 52, a display 53, and a control unit 54.

The touch panel 51 is a panel that is touched by a target of fingerprint collection. When the rotated fingerprint image is collected, the target may move a finger to rotate on the touch panel 51. The scanner unit 52 is disposed below the touch panel 51 and scans the fingerprint image of the finger that touches the touch panel 51. The fingerprint image scanned by the scanner unit 52 is acquired by the fingerprint image acquisition unit 110 (see FIG. 2, etc.).

The display 53 is configured to display the fingerprint image scanned by the scanner unit 52. The display 53 may also be configured to display information about the type of the finger estimated from the scanned rotated fingerprint image. The display 53 may further be configured to display various types of information, such as the registration error information described in the second example embodiment (see FIG. 4 and FIG. 5), the information indicating the designated finger and the collection error information described in the third example embodiment (see FIG. 6 and FIG. 7), and the missing information described in the fifth example embodiment (see FIG. 10 and FIG. 11).

The control unit 54 is a controller including, for example, the processor 11, and is configured to realize the components provided in the estimation apparatus 10 according to each of the above example embodiments. Specifically, the control unit 54 may realize the functions of each of the fingerprint image acquisition unit 110, the finger type estimation unit 120, the fingerprint image storage unit 130, the registered finger type determination unit 140, the finger type designation unit 150, the designated finger type determination unit 160, the fingerprint image registration unit 170, the missing finger type determination unit 180, the image quality determination unit 190, and the substitute registration unit 200.

### (Technical Effect)

Next, a technical effect obtained by the estimation apparatus 10 according to the seventh example embodiment will be described.

As described in FIG. 15, according to the estimation apparatus 10 in the seventh example embodiment, it is possible to estimate the type of the finger included in the rotated fingerprint image with high accuracy, by collecting the rotated fingerprint image of the target. This estimation apparatus 10 is applicable, for example, as an apparatus that is used when fingerprints are registered by the police in a fingerprint matching/identification system. It is also applicable as an apparatus that collects fingerprints at immigration at an airport, or the like.

### Other Application Examples

The estimation apparatus 10 according to each of the above example embodiments is applicable, for example, to portable terminals such as smartphones, general home appliances, and the like.

For example, in a case where a shortcut function of a portable terminal is assigned to a particular finger, it is necessary to register the finger in advance (i.e., to associate the type of the finger with the function to perform). In the estimation apparatus 10 according to the present example embodiment, however, the type of a touching finger may be estimated, and it is thus possible to eliminate the trouble of registering the finger in advance.

For example, a plurality of actions in various applications may be assigned to respective fingers. Specifically, in a book viewing application, touching with an index finger may cause a “page turn” function to be performed, and touching with a middle finger may cause a “magnifying glass” function to be performed. In a video playback application, touching with the index finger could may cause a “play” function to be performed, and touching with the middle finger may cause a “fast forward” function to be performed. Alternatively, when a security lock of a portable terminal is released, a function corresponding to an operating finger may be started after unlocking.

It is also possible to treat a long press on a screen with a finger that is normally not used to operate the portable terminal, or similar actions, as a default function. For example, in a case where the screen is long pressed with a left little finger, emergency calls such as 110 and 119, phone numbers in Japan, may be made, or frequently called numbers may be automatically dialed.

In application to general home appliances, an answer corresponding to the type of a touching finger may be selected to a question asked during initial setup. Specifically, the index finger may be used to touch the screen to indicate “yes”, and the middle finger to indicate “no.” Such a function exhibits a remarkable effect in an apparatus for which it is desired to operate without looking at the screen, such as car navigation.

In addition, by installing it in a remote control for a television or the like, it is possible to realize channel forwarding or volume adjustment using a particular finger. by installing it in an electronic piano, it is possible to realize a fingering practice function when playing a song.

A processing method that is executed on a computer by recording, on a recording medium, a program for allowing the configuration in each of the example embodiments to be operated so as to realize the functions in each example embodiment, and by reading, as a code, the program recorded on the recording medium, is also included in the scope of each of the example embodiments. That is, a computer-readable recording medium is also included in the range of each of the example embodiments. Not only the recording medium on which the above-described program is recorded, but also the program itself is also included in each example embodiment.

The recording medium to use may be, for example, a floppy disk (registered trademark), a hard disk, an optical disk, a magneto-optical disk, a CD-ROM, a magnetic tape, a nonvolatile memory card, or a ROM. Furthermore, not only the program that is recorded on the recording medium and that executes processing alone, but also the program that operates on an OS and that executes processing in cooperation with the functions of expansion boards and another software, is also included in the scope of each of the example embodiments. In addition, the program itself may be stored in a server, and a part or all of the program may be downloaded from the server to a user terminal. The program may be provided to a user in a form of Saas (Software as a Service), for example.

### Supplementary Notes

The example embodiments described above may be further described as, but not limited to, the following Supplementary Notes below.

### (Supplementary Note 1)

An estimation apparatus according to Supplementary Note 1 is an estimation apparatus including: an acquisition unit that acquires a rotated fingerprint image collected by rotating a finger; and an estimation unit that estimates a type of a finger included in the rotated fingerprint image, by inputting the acquired rotated fingerprint image into a learned model.

### (Supplementary Note 2)

An estimation apparatus according to Supplementary Note 2 is the estimation apparatus according to Supplementary Note 1, further including: a storage unit that stores the rotated fingerprint image in association with a type of a finger; and a first output unit that outputs information indicating that the type of the finger stored in the storage unit is incorrect, in a case where the type of the finger estimated by the estimation unit from the rotated fingerprint image is different from the type of the finger stored in association with the rotated fingerprint image.

### (Supplementary Note 3)

An estimation apparatus according to Supplementary Note 3 is the estimation apparatus according to Supplementary Note 1 or 2, further including: a designation unit that designates a type of a finger from which the rotated fingerprint image is to be collected; and a second output unit that outputs information indicating that the type of the finger from which the rotated fingerprint image is to be collected, is incorrect, in a case where the type of the finger estimated by the estimation unit from the rotated fingerprint image is different from the type of the finger designated by the designation unit.

### (Supplementary Note 4)

An estimation apparatus according to Supplementary Note 4 is the estimation apparatus according to any one of Supplementary Notes 1 to 3, further including: a registration unit that registers the collected rotated fingerprint image in association with the type of the finger estimated by the estimation unit.

### (Supplementary Note 5)

An estimation apparatus according to Supplementary Note 5 is the estimation apparatus according to Supplementary Note 4, further including: a third output unit that outputs information prompting collection of the rotated fingerprint image of a missing finger, in a case where there is a shortage of the type of the finger of the rotated fingerprint image registered by the registration unit.

### (Supplementary Note 6)

An estimation apparatus according to Supplementary Note 6 is the estimation apparatus according to any one of Supplementary Notes 1 to 5, further including: a determination unit that determines whether or not quality of the rotated fingerprint image satisfies a predetermined criterion; and a substitute registration unit that registers a planar fingerprint image of a same type of a finger as that of the rotated fingerprint image, instead of the rotated fingerprint image, in a case where the quality of the rotated fingerprint image does not satisfy the predetermined criterion.

### (Supplementary Note 7)

An estimation apparatus according to Supplementary Note 7 is the estimation apparatus according to any one of Supplementary Notes 1 to 6, further including:
a storage unit that stores the rotated fingerprint image in association with a type of a finger; and a substitute registration unit that registers a planar fingerprint image of a same type of a finger as that of the rotated fingerprint image, instead of the rotated fingerprint image, in a case where the type of the finger estimated by the estimation unit from the rotated fingerprint image is different from the type of the finger stored in association with the rotated fingerprint image.

### (Supplementary Note 8)

An estimation apparatus according to Supplementary Note 8 is the estimation apparatus according to any one of Supplementary Notes 1 to 7, further including: a touch panel that is touched by a target with a finger; and a scanner that scans the finger with which the touch panel is touched to acquire the rotated fingerprint image.

### (Supplementary Note 9)

An estimation apparatus according to Supplementary Note 9 is the estimation apparatus according to any one of Supplementary Notes 1 to 8, further including: a display that displays at least one of the rotated fingerprint image and information about the type of the finger estimated from the rotated fingerprint image.

### (Supplementary Note 10)

An estimation method according to Supplementary Note 10 is an estimation method that is executed by at least one computer, the estimation method including: acquiring a rotated fingerprint image collected by rotating a finger; and estimating a type of a finger included in the rotated fingerprint image, by inputting the acquired rotated fingerprint image into a learned model.

### (Supplementary Note 11)

A computer program according to Supplementary Note 11 is a computer program that allows at least one computer to execute an estimation method, the estimation method including: acquiring a rotated fingerprint image collected by rotating a finger; and estimating a type of a finger included in the rotated fingerprint image, by inputting the acquired rotated fingerprint image into a learned model.

### (Supplementary Note 12)

A recording medium according to Supplementary Note 11 is a recording medium on which a computer program that allows at least one computer to execute an estimation method is recorded, the estimation method including: acquiring a rotated fingerprint image collected by rotating a finger; and estimating a type of a finger included in the rotated fingerprint image, by inputting the acquired rotated fingerprint image into a learned model.

The present disclosure is allowed to be changed, if desired, without departing from the essence or spirit of this disclosure which can be read from the claims and the entire specification. An estimation apparatus, an estimation method, and a recording medium with such changes are also intended to be within the technical scope of the present disclosure.

To the extent permitted by law, this application is based on and claims priority to Japanese Patent Application No. 2023-020932 filed on Feb. 14, 2023, the disclosure of which is incorporated herein in its entirety by reference. Furthermore, to the extent permitted by law, all published documents and papers cited in the present specification are incorporated herein.

### DESCRIPTION OF REFERENCE CODES

10 Estimation apparatus
11 Processor
51 Touch panel
52 Scanner unit
53 Display
54 Control unit
110 Fingerprint image acquisition unit
120 Finger type estimation unit
130 Fingerprint image storage unit
140 Registered finger type determination unit
150 Finger type designation unit
160 Designated finger type determination unit
170 Fingerprint image registration unit
180 Missing finger type determination unit
190 Image quality determination unit
200 Substitute registration unit

What is claimed is:

1. An estimation apparatus comprising:
at least one memory that is configured to store instructions; and
at least one processor that is configured to execute the instructions to:
acquire a rotated fingerprint image collected by rotating a finger; and
estimate a type of a finger included in the rotated fingerprint image, by inputting the acquired rotated fingerprint image into a learned model.

2. The estimation apparatus according to claim 1, wherein the at least one processor is configured to execute the instructions to:
store the rotated fingerprint image in association with a type of a finger; and
output information indicating that the type of the finger stored is incorrect, in a case where the type of the finger estimated from the rotated fingerprint image is different from the type of the finger stored in association with the rotated fingerprint image.

3. The estimation apparatus according to claim 1, wherein the at least one processor is configured to execute the instructions to:
designate a type of a finger from which the rotated fingerprint image is to be collected; and
output information indicating that the type of the finger from which the rotated fingerprint image is to be collected, is incorrect, in a case where the type of the finger estimated from the rotated fingerprint image is different from the type of the finger designated

4. The estimation apparatus according to claim 1, wherein the at least one processor is configured to execute the instructions to:
register the collected rotated fingerprint image in association with the type of the finger estimated.

5. The estimation apparatus according to claim 4, wherein the at least one processor is configured to execute the instructions to:
output information prompting collection of the rotated fingerprint image of a missing finger, in a case where there is a shortage of the type of the finger of the rotated fingerprint image registered.

6. The estimation apparatus according to claim 1, wherein the at least one processor is configured to execute the instructions to:
determine whether or not quality of the rotated fingerprint image satisfies a predetermined criterion; and
register a planar fingerprint image of a same type of a finger as that of the rotated fingerprint image, instead of the rotated fingerprint image, in a case where the quality of the rotated fingerprint image does not satisfy the predetermined criterion.

7. The estimation apparatus according to claim 1, further comprising:
a touch panel that is touched by a target with a finger; and
a scanner that scans the finger with which the touch panel is touched to acquire the rotated fingerprint image.

8. The estimation apparatus according to claim 1, further comprising:
a display that displays at least one of the rotated fingerprint image and information about the type of the finger estimated from the rotated fingerprint image.

9. An estimation method that is executed by at least one computer, the estimation method comprising:
acquiring a rotated fingerprint image collected by rotating a finger; and
estimating a type of a finger included in the rotated fingerprint image, by inputting the acquired rotated fingerprint image into a learned model.

10. A non-transitory recording medium on which a computer program that allows at least one computer to execute an estimation method is recorded, the estimation method including:
acquiring a rotated fingerprint image collected by rotating a finger; and
estimating a type of a finger included in the rotated fingerprint image, by inputting the acquired rotated fingerprint image into a learned model.
